(19) United States
(12) Patent Application Publication
(10) Pub. No.: US 2015/0105845 A1
(43) Pub. Date: Apr. 16, 2015

(54) ADJUSTABLE HEATING/COOLING PACK

(71) Applicant: Edward Lee Duckworth, JR., Chicago, IL (US)

(72) Inventor: Edward Lee Duckworth, JR., Chicago, IL (US)

(21) Appl. No.: 14/055,431

(22) Filed: Oct. 16, 2013

(51) Int. Cl. A61F 7/08

(52) U.S. Cl. CPC A61F 7/08; A61F 2007/0042
USPC 607/112; 607/114

(57) ABSTRACT

The present device generally relates to an adjustable heating/cooling pack. The heating/cooling pack is especially suitable for use on an injured or sore leg or arm of a user. The heating/cooling pack may first be placed in a freezer or refrigerator (or microwave if heat is desired) and removed prior to use. The device has a securing means which allows it to remain in a rolled up tube configuration having a closed environment prior to use. As a result, the device remains cool or warm for a longer period of time than other heating/cooling packs. In addition, items may be stored and carried inside the device while the device is in the closed configuration (third position).

### CROSS REFERENCE TO RELATED APPLICATION

The following application is based on and claims the priority benefit of U.S. Provisional Application Ser. No. 61/714,938 filed on Oct. 17, 2012, currently co-pending; the entire contents of which are incorporated by reference.

### BACKGROUND OF THE DEVICE

The present device generally relates to an adjustable heating/cooling pack. The heating/cooling pack is especially suitable for use on an injured or sore leg or arm of a user. The heating/cooling pack may first be placed in a freezer or refrigerator (or microwave if heat is desired) and removed prior to use. The device has a securing means which allows it to remain in a rolled up tube configuration having a closed environment prior to use. As a result, the device remains cool or warm for a longer period of time than other heating/cooling packs. In addition, items may be stored and carried inside the device while the device is in the closed configuration (third position).

Over the years numerous cooling packs have been developed. For example, U.S. Pat. No. 5,113,348 to Mayn discloses a hot or cold pack with a main body portion and four radially extending portions attached thereto and integral therewith. The radially extending portions may have a width greater at their periphery than at the joining to the main body portion. The hot or cold pack is particularly useful for application to a curved contour such as breast, knee, ankle, shoulder or other body area to relieve post operative or post trauma pain and inflammation.

U.S. Pat. No. 4,552,149 to Tatsuki discloses a head cooling implement capable of covering the human skull like a cap for medical treatment. It comprises a main body consisting of a cooling piece for covering the top of the head and a plurality of cooling pieces radially arranged around the first-mentioned cooling piece for covering the front, sides and back of the head, each cooling piece being in the form of a sealed bag containing a coolant; a stretchable belt applied to the chin so as to bring the head top cooling piece of the main body into close contact with the top of the head; another stretchable belt adapted to bring the other cooling pieces of the main body into close contact with the skull at suitable places there around; and a heat insulating cap to be put over the main body.

However, these heating/cooling packs fail to disclose a heating/cooling pack which stores heat or cold in an easy and efficient manner. A need, therefore, exists for an improved heating/cooling pack for use in connection with injury or other health related conditions.

### SUMMARY OF THE INVENTION

The present device generally relates to an adjustable heating/cooling pack. The heating/cooling pack is especially suitable for use on an injured or sore leg or arm of a user. The heating/cooling pack may first be placed in a freezer or refrigerator (or microwave if heat is desired) and removed prior to use. The device has a securing means which allows it to remain in a rolled up tube configuration having a closed environment prior to use. As a result, the device remains cool or warm for a longer period of time than other heating/cooling packs. In addition, items may be stored and carried inside the device while the device is in the closed configuration (third position).

An advantage of the present device is to provide a heating/cooling pack which is easy to use.

Yet another advantage of the present device is to provide a heating/cooling pack which efficiently retains heat or cold.

Still another advantage of the present device is to provide a heating/cooling pack which completely seals and encloses the heating or cooling portion.

Another advantage of the present device is to provide a heating/cooling pack which allows one to carry items within the interior of the heating/cooling pack.

And another advantage of the present device is to provide a heating/cooling pack which is cost efficient.

Still another advantage of the present device is to provide a heating/cooling pack which is easy to transport.

For a more complete understanding of the above listed features and advantages of the adjustable heating/cooling pack reference should be made to the following detailed description of the preferred embodiments and to the accompanying drawings.

### BRIEF DESCRIPTION OF THE DRAWING

FIG. 1 illustrates a front plan view of the heating/cooling pack device in a flat (first position) orientation.

FIG. 2 illustrates a perspective view of the heating/cooling pack wherein the heating/cooling pack is in a partially rolled up configuration.

FIG. 3 illustrates a side perspective view of the heating/cooling pack wherein the device is in the third orientation for transportation.

FIG. 4 illustrates a top plan view of the heating/cooling pack in the third orientation for transportation.

FIG. 5 illustrates a side perspective view of the heating/cooling pack in the second orientation and secured on the knee of a person.

FIG. 6 illustrates an embodiment of the heating/cooling pack in a rolled up orientation wherein the heating cooling pack as a strap and wherein an extension piece is secured to an end of the device.

FIG. 7 illustrates a view of the interior of the heating/cooling pack through an extension piece in an embodiment of the device.

FIG. 8 illustrates a front view of an embodiment of the heating/cooling pack in the flat first orientation.

FIG. 9 illustrates a back view of an embodiment of the heating/cooling pack in the flat first orientation.

FIG. 10 illustrates the heating cooling/pack wherein the generally circular flaps are removed and wherein extension pieces are secured to the top edge and the bottom edge.

### DETAILED DESCRIPTION OF THE PREFERRED EMBODIMENTS

The present device generally relates to an adjustable heating/cooling pack. The heating/cooling pack is especially suitable for use on an injured or sore leg or arm of a user. The heating/cooling pack may first be placed in a freezer or refrigerator (or microwave if heat is desired) and removed prior to use. The device has a securing means which allows it to remain in a rolled up tube configuration having a closed environment prior to use. As a result, the device remains cool or warm for a longer period of time than other heating/cooling packs. In addition, items may be stored and carried inside the device while the device is in the closed configuration (third position).

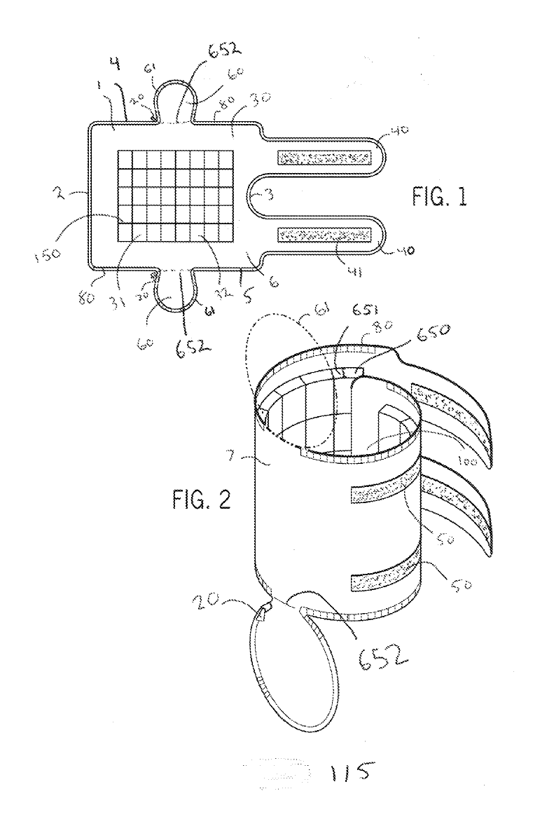

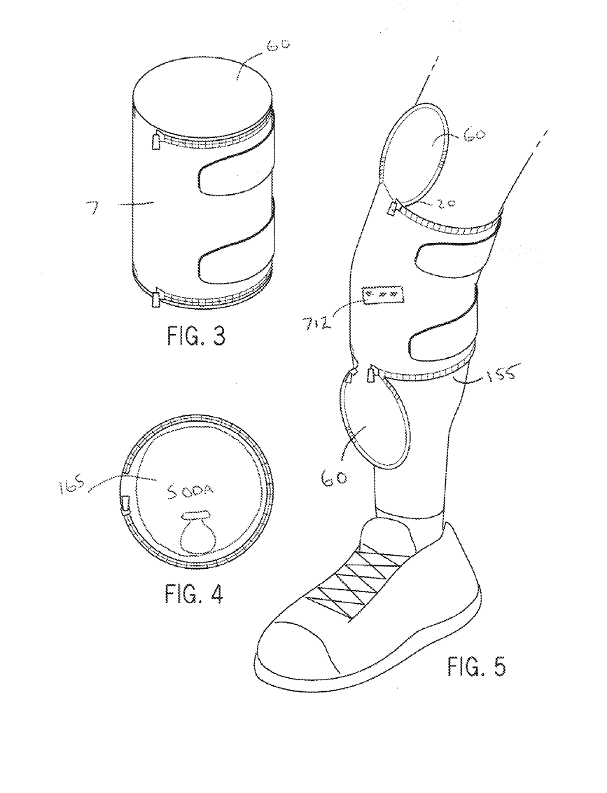

Referring now to FIG. 1, in an embodiment, the heating/cooling pack 1 may have a first side 2, a second side 3, a top edge 4, a bottom edge 5, a front 6, a back 7 (FIG. 2) and an interior 100 (FIGS. 2 and 7). The heating/cooling pack 1 may be transformed through various orientations. More specifically, in the first orientation (FIG. 1) the device 1 may be generally flat and may be used by a person, for example, to treat a sore or otherwise injured part of the body. In a second orientation (FIG. 5) the device may be wrapped around, for example, the knee of a person to treat an injury. In a third orientation (FIG. 3), the device 1 may be in an orientation suitable for being transported prior to use or in an orientation suitable to carry and heat or cool an item, such as a drink. In this third orientation embodiment, the device 1 may generally resemble a sealed tube. Further, in the sealed tube orientation, the device 1 acts as a thermos.

The heating/cooling pack 1 may, for example, be placed in the microwave or in hot water for heating or may be placed in a freezer or refrigerator for cooling. After the heating/cooling pack 1 has reached the desired temperature (either hot or cold) the device 1 may be transported in the third orientation for eventual use in the second orientation on the body of a person (or first orientation if the user simply places the device 1 flush on the skin over an injury).

In an embodiment (FIG. 1), the heating/cooling pack 1 may have a support element 30 and a heating/cooling element 31. The support element 30 may be, for example, constructed of plastic, rubber, cloth or the like such that the support element 30 may bend easily but may also be durable. It should be noted that the support element 30 of the heating/cooling pack 1 may be constructed of any suitable material to accomplish the desired function.

The heating/cooling element 31 of the device 1 may, for example, resemble a grid and may be constructed of largely square gel elements 32 having a plurality of generally perpendicular creases 150 located between the gel elements 32. The heating/cooling element 31 portion of the heating/cooling pack 1 may be made from, for example, a typical gel as is common in gel packs. In an embodiment, the heating/cooling element 31 is generally square-shaped so as to cover a large portion of an item 165 (FIG. 4) or body part to be heated or cooled.

In an embodiment, the square gel elements 32 may have a side 650 having an extended height 651 above the main surface of the front 6 of the support element 30. The extended height 651 of the square gel elements 32 may allow the square gel elements 32 to more easily contact an item 165 or body part to be cooled or warmed.

The creases 150 may allow the surface of the heating/cooling elements 31 to easily be bent into various orientations so as to better surround an item 165 (such as a drink) to be heated or cooled or a body part (such as a knee or elbow) to be treated. The heating/cooling element 31 of the device 1 may be located on the front 6 of the support element 30; occupying a large percentage of the front 6 of the device 1. While the heating cooling element 31 may be located on the front 6 of the device 1, the back 7 of the device 1 may be constructed of the structural element 30 and the back 7 of the device 1 may, in an embodiment, lack the heating/cooling element 31.

The second side 3 side of the heating/cooling pack 1 may have a securing means 40. FIG. 1 illustrates two securing means 40 extending from the second side 3; however, it should be understood that any number of securing means 40 may be implemented. The securing means 40 may resemble elongated strips which extend out away from the body of the support element 30. Located on each of the securing means 40 may be a hook and loop fastener strip 41. The hook and loop fastener strip 41 may be slightly smaller in size than the securing means 40.

In an embodiment, a first generally circular flap 60 may be located at the top edge 4 and a second generally circular flap 60 may be located at the bottom edge 5 of the heating/cooling pack 1. In an embodiment, the generally circular flap 60 is located in the center of the top edge 4 and the center of the bottom edge 5. The circular flap 60 may be connected to the front 6 of the device 1 at a crease 652. The crease 652 may allow the generally circular flap 60 to bend from a first position (generally parallel to the front 6 of the device 1) into a second position (generally perpendicular orientation with respect to the front 6 of the device 1).

The generally circular flap 60 may be partially connected to the main body of the heating/cooling pack 1. Along a perimeter 61 of the generally circular flaps 60 may be, for example, a zipper 20 (or other securing means). The zipper 20 (or other securing means) may connect the generally flap 60 of the device 1 to an elongated straight perimeter 80 located on the top 4 and bottom 5 of the main body of the heating/cooling pack 1.

In an embodiment, a second securing means 50 may be located on the back side 7 of the heating/cooling pack 1 (see FIG. 2). The second securing means 50 may be the corresponding strip which mates with the hook and loop fastener strip 41 of the securing means 40. Preferably, the second securing means 50 is greater in size and length than the hook and loop fastener strip 41 such that a user may tighten or loosen the device 1 depending on where on the second securing means 50 the hook and loop fastener strip 41 is secured.

During use in the first or second orientation, the user may strap the heating/cooling pack 1 around, for example, the knee, leg, elbow or arm of the user. More specifically, the front side 6 of the heating/cooling pack 1 may be secured around, for example, the leg 155 (FIG. 5) of the user such that the heating/cooling element 31 is in direct contact with the skin. The grid of largely square elements 32 may contact, for example, a sore or otherwise injured part of a person.

When removed from the freezer or refrigerator (or heated if the user wishes the device 1 to be warm), the user may keep the heating/cooling pack 1 in the third orientation (FIGS. 3 and 4) wherein the generally circular flap 60 at the top edge 4 of the device 1 is secured to the top perimeter 80 of the device 1 and the generally circular flap 60 at the bottom edge 5 of the device 1 is secured to the bottom perimeter 80 of the device 1 therein forming a completely sealed tube. As a result of being placed in the tube-shaped third orientation (FIG. 3), the heating/cooling pack 1 retains its cool or warm temperature for a much longer time than other heating cooling packs as a result of reduced (or no) surface area of the gel 32 being exposed to the outside air. Further, a person may store an item 165 (FIG. 4) such as a drink, soup, food or the like in the interior 100 of the device 1 while the device 1 is in the rolled up closed third orientation. As a result, the device 1 may not only act as a heating/cooling pack 1, but may also act as a thermos prior to use.

Referring now to FIG. 5, in an embodiment, the device 1 may be worn on a person. In this orientation, the generally circular flaps 60 may not be secured to the elongated straight perimeter 80 of the top edge 4 and the bottom edge 5 of the device 1, but instead the generally circular flaps 60 may freely move and may rest in a generally parallel orientation with respect to the front 6 of the device 1. In this embodiment, the generally circular flaps 60 may provide additional protection to, for example, a shin or leg of a person by covering a portion of the user's body and protecting the same from injury.

In an embodiment, the back 7 of the device 1 may have a generally reflective portion 712. The generally reflective portion 712 may allow a user to wear the device 1 in reduced light therein increasing the user's visibility to vehicles so as to increase safety of the user while, for example, jogging.

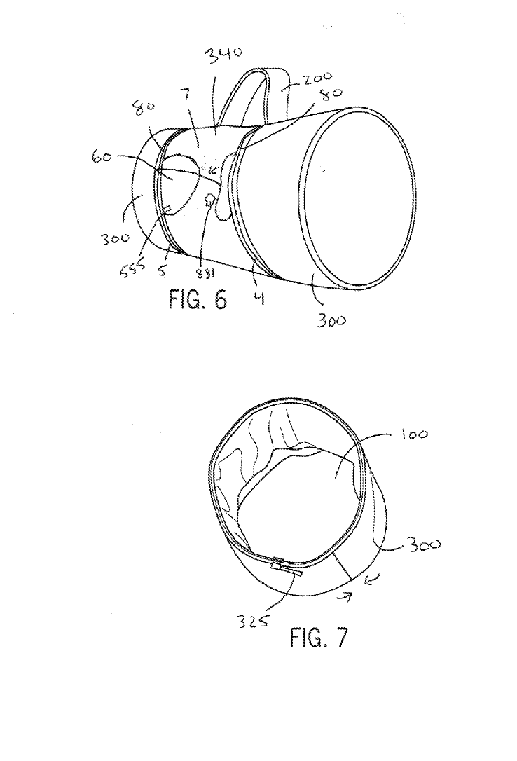

Referring now to FIG. 6, in an embodiment, the heating/cooling pack 1 may have a strap 200. The strap 200 may allow the device 1 to be carried easily. The strap 200 may be permanently secured to the back 7 of the device 1 such that the strap 200 may be used even when the device 1 is rolled up into the third orientation.

In an embodiment, the device 1 may have at least one extension piece 300 (FIGS. 6 and 7). The extension pieces 300 may be generally rectangular (FIG. 7 illustrated the extension piece 300 rolled up). The extension piece 300 may have a width which is identical to the first side 2 and the second side 3 of the device 1 such that the extension piece 300 may be correspondingly secured to the first side 2 and the second side 3 without the extension piece 300 extending beyond the top edge 4 or bottom edge 5 of the device 1 and without the extension piece 300 being too short to reach from the top edge 4 to the bottom edge 5.

In an embodiment, the extension pieces 300 may be secured to the top edge 4 and/or the bottom edge 5 of the support element 30 at the elongated perimeter 80 of the device 1. The extension pieces 300 may allow the device 1 to be used to heat or cool a larger area (suitable to secure larger items 165 or heat of cool larger portions of a body). A zipper 325 may be used to temporarily secure the extension pieces 300 to the main body 340 of the device 1.

When the extension pieces 300 are utilized, the generally circular flaps 60 may fold backward back onto the back 7 of the support element 30 and may be secured to the back 7 by, for example, a securing means 881. The securing means 881 may be, for example, a hook a loop fastener system. Securing the generally circular flaps 60 to the back 7 of the support element 30 when the extension pieces 300 are used prevents the generally circular flaps 60 from moving freely. FIG. 6 illustrates one of the generally circular flaps 60 secured to the back 7 and the other in the process of being secured to the back 7. In an embodiment, the generally circular flaps 60 may be removed from the support element 30 when the device 1 utilizes the extension pieces 300. More specifically, FIG. 10 illustrates the generally circular flaps 60 removed via a zipper 555.

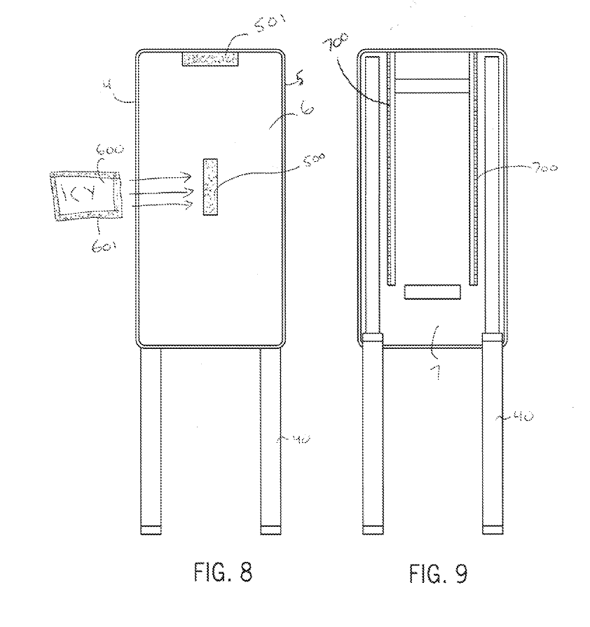

Referring now to FIG. 8, in an embodiment, a generally rectangular first hook and loop fastener 500 and second hook and loop fastener 501 may be located on the front 6 of the device 1. The first hook and loop fastener 500 may be used to temporarily secure, for example, a removable heating or cooling pack 600 wherein the heating and cooling pack 600 has a corresponding hook and loop fastener strip 601 which temporarily mates with the first hook and loop fastener 500.

In an embodiment, the first hook and loop fastener 500 may be located generally near the center of the front 6 in a generally vertical position with respect to the top 4 and the bottom 5 of the device 1. The first hook and loop fastener 500 may temporarily secure the heating or cooling pack 600 to the device 1 such that the heating and cooling pack 600 may be therein placed next to an item 165 or body part being heated or cooled. A user may therein switch out the heating or cooling pack 600 without the need to completely undo the device 1. The second hook and loop fastener 501 on the front 4 of the device 1 may be used to secure the device 1 in the generally useful second or third orientation.

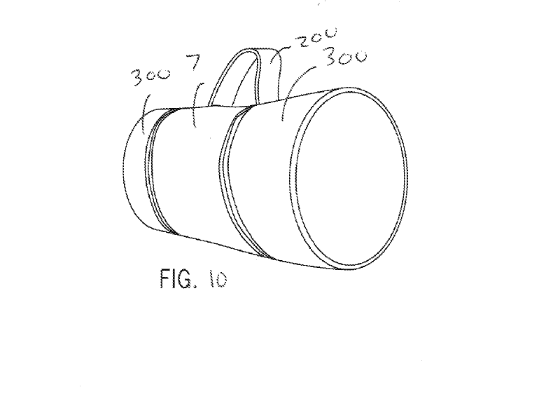

Referring now to FIG. 9, in an embodiment, a first and second zipper 700 may be located on the back 7 of the device 1. The first and second zipper 700 may be generally parallel with respect to one another and may correspondingly mate with the zipper 325 (FIG. 7) of the extension pieces 300. In an embodiment, the first and second zipper 700 do not extend the entire length of the device 1. More specifically, the first and second zipper 700 may contact one side of the device 1 but may not contact the opposing side of the device 1. The first and second zipper 700 of the back 7 of the device 1 may not extend across the entire back 7 so that a portion of the side wherein the first and second zipper 700 do not contact may be smoothly folded under the side wherein the first and second zipper 700 does contact the side without a portion of the first and second zipper causing a bump when worn.

Although embodiments of the present invention are shown and described therein, it should be understood that various changes and modifications to the presently preferred embodiments will be apparent to those skilled in the art. Such changes and modifications may be made without departing from the spirit and scope of the present invention and without diminishing its attendant advantages. It is, therefore, intended that such changes and modifications be covered by the appended claims.

1. An adjustable heating/cooling pack comprising:
a flexible support element having a top edge, a bottom edge, a front, a back, a first side and a second side wherein a first securing mechanism is located at the top edge and wherein a second securing mechanism is located at the bottom edge;
a first generally circular flap having a generally circular edge located at the top edge of the support element wherein the first generally circular flap is connected to the top edge of the support element at a first crease and wherein the first crease at the top of the support element allows the first generally circular flap to pivot with respect to the support element;
a second generally circular flap having a generally circular edge located at the bottom edge of the support element wherein the second generally circular flap is connected to the bottom edge of the support element at a second crease and wherein the second crease allows the second generally circular element at the bottom edge of the support element to pivot with respect to the support element;
a third securing mechanism located on the generally circular edge of the first generally circular flap;
a fourth securing mechanism located on the generally circular edge of the second generally circular flap; and
wherein the third securing mechanism is temporarily secured to the first securing mechanism and wherein the fourth securing mechanism is temporarily secured to the second securing mechanism and wherein the heating/cooling pack moves from a generally flat orientation into a generally cylindrical tube orientation.

2. The adjustable heating/cooling pack of claim 1 wherein the first, second, third or fourth securing mechanism is a zipper.

3. The adjustable heating/cooling pack of claim 1 further comprising:
a heating/cooling element located on the front surface of the support element.

4. The adjustable heating/cooling pack of claim 3 further comprising:
a side of the heating/cooling element wherein the side of the heating/cooling element is elevated off the surface of the front of the support element.

5. The adjustable heating/cooling pack of claim 3 wherein the heating/cooling element is made of a plurality of square-shaped units forming a grid and wherein a cease exists between any two of the square-shaped units and allows the heating/cooling pack to bend easily.

6. The adjustable heating/cooling pack of claim 3 wherein the heating/cooling element is made of a gel.

7. The adjustable heating/cooling pack of claim 1 further comprising:
a strap located on the second side of the support element wherein the strap on the second side wraps around and is secured to a portion of the back of the first side of the support element.

8. The adjustable heating/cooling pack of claim 1 further comprising:
a hook and loop fastener located on the front of the support element wherein the hook and loop fastener temporarily secures an ice pack or heating pack having a corresponding hook and loop fastener strip.

9. The adjustable heating/cooling pack of claim 1 wherein the heating/cooling pack is substantially sealed in the generally cylindrical tube orientation and wherein the heating/cooling pack acts as a thermos in the generally cylindrical tube orientation and wherein the heating/cooling pack stores and warms or cools an item.

10. The adjustable heating/cooling pack of claim 1 further comprising:
a reflective element located on the back of the support element wherein the reflective element allows for increased visibility of a person wearing the heating/cooling pack.

11. The adjustable heating/cooling pack of claim 1 further comprising:
an extension piece temporarily secured to the first side and/or the second side of the support element wherein the extension piece allows the heating/cooling pack to have a longer top edge and a longer bottom edge.

12. The adjustable heating/cooling pack of claim 1 further comprising:
a first and a second zipper on the back of the support element wherein the first and second zipper are parallel to each other and wherein the first and second zipper touch the second side of the support element but do not touch the first side of the support element.

13. An adjustable heating/cooling pack comprising:
a flexible support element having a top edge, a bottom edge, a front, a back, a first side and a second side wherein a first securing mechanism is located at the top edge and wherein a second securing mechanism is located at the bottom edge;
a first generally circular flap having a generally circular edge located at the top edge of the support element wherein the first generally circular flap is connected to the top edge of the support element at a first crease and wherein the first crease at the top of the support element allows the first generally circular flap to pivot with respect to the support element;
a second generally circular flap having a generally circular edge located at the bottom edge of the support element wherein the second generally circular flap is connected to the bottom edge of the support element at a second crease and wherein the second crease allows the second generally circular element at the bottom edge of the support element to pivot with respect to the support element;
a third securing mechanism located on the generally circular edge of the first generally circular flap;
a fourth securing mechanism located on the generally circular edge of the second generally circular flap; and
wherein the adjustable heating/cooling pack moves from a generally flat first orientation into a functional second orientation wherein the heating/cooling device is secured onto a person in the functional second orientation.

14. The adjustable heating/cooling pack of claim 13 further comprising:
a strap on the second end wherein the strap on the second end wraps partially around a portion of a person's body and is secured to the back of the support element near the first side of the support element so that the adjustable heating/cooling pack fully surrounds a portion of the person's body and is secured into place by the strap.

15. The adjustable heating/cooling pack of claim 13 wherein the first generally circular flap extends from the top edge of the support element and covers and protects a portion of the person's body and wherein the generally first circular flap does not completely surround the portion of the person's body.

16. The adjustable heating/cooling pack of claim 13 further comprising:
a securing mechanism in the first crease wherein the securing mechanism allows for the temporarily addition or removal of the first generally circular flap from the support element.

17. The adjustable heating/cooling pack of claim 13 further comprising:
a securing mechanism on the back of the support element wherein the securing mechanism on the back of the support element temporarily secures the generally circular flap to the back of the support element.
